(19) United States
(12) Patent Application Publication
(10) Pub. No.: US 2016/0370265 A1
(43) Pub. Date: Dec. 22, 2016

(54) METHOD FOR ISOLATING EXOSOMES

(71) Applicants: BIOMÉRIEUX, Marcy-l'etoile (FR); HOSPICES CIVILS DE LYON, Lyon (FR)

(72) Inventors: Catherine OTT, Saint-Priest (FR); François MALLET, VILLEURBANNE (FR); Laurence GENERENAZ, Saint-Priest (FR)

(21) Appl. No.: 15/100,931

(22) PCT Filed: Dec. 3, 2014
(86) PCT No.: PCT/FR2014/053143

§ 371 Date: Jun. 1, 2016

(30) Foreign Application Priority Data
Dec. 3, 2013 (FR) 13/62021

(51) Int. Cl. G01N 1/34; G01N 33/50

(52) U.S. Cl. CPC G01N 1/34; G01N 33/5076; G01N 2800/52

(57) ABSTRACT

The invention concerns a method for isolating exosomes from a biological liquid, comprising at least the following two successive steps of affinity purification:
a) a first step using at least one specific anti-ligand of a generic ligand of the exosomes, so as to obtain a population P of exosomes, said exosomes being separated from said anti-ligand, and
b) a second step, applied on the population P of exosomes, using at least one specific anti-ligand of a ligand characteristic of a subpopulation SP of exosomes, so as to obtain said subpopulation SP of exosomes, said exosomes being separated or not from said anti-ligand.
   as well as the applications of such method.

### TECHNICAL FIELD

The present invention concerns a method for isolating exosomes from a biological sample, and the use of this method as a tool in the characterization of the exosomes present in said sample, as well as in the diagnosis and prognosis of a pathology and/or of the clinical stage of a pathology, but also in monitoring the evolution of a pathology, whether treated or not, in a human or an animal.

### BACKGROUND

The exosomes are membrane vesicles of a 40-120 nm diameter, secreted in vivo by different cell types. Because of their origin and their biogenesis, the exosomes reflect the content and the normal or pathological physiological condition of the cells from which they are derived. They are found in numerous biological liquids, such as blood, plasma, serum, urine, saliva, cerebrospinal fluid (CSF), lymph, bile, bronchoalveolar lavages, semen, synovial fluid, amniotic fluid, breast milk, malignant ascites fluids, . . . .

The secretory process is a very active process for proliferating cells such as cancerous cells. They contain nucleic and proteinic markers of tumoral cells, from which they are secreted, and therefore, they are considered as reservoirs of potential new biomarkers of cancer. As such, their study presents a growing interest for their the scientific community.

The major limitation in their study lies in obtaining purified and sufficiently enriched preparations, from the different aforementioned biological fluids, with the current techniques available in the related art. Indeed, the literature highlights the complexity of the assessment of exosomes because of contaminated preparations, in particular by proteins, microsomal fractions or organelles co-purified by techniques such as ultracentrifugation or nanofiltration, or because of preparations too little concentrated in exosomes to be analyzed, derived from immunological separation techniques. To date, even the combination of these techniques has not proved to be fully satisfactory.

Most of the works carried out with the aim of characterizing the exosomes have been undertaken on cultures of malignant cells. These have been subjected to techniques of purification of the exosomes secreted by the cells during development, and proteomic profiles of the isolated exosomes are determined. Thus, according to the article S. Mathivanan et al., Mol Cell Proteomics. 2010 February; 9(2):197-208, the authors have isolated exosomes from a cell line of the human carcinoma of the colon, LIM1215. The culture medium has first been subjected to a first series of ultracentrifugations in order to separate therefrom a population of particles of a 40-100 nm size, which has subsequently been involved in an immunopurification with a humanized antibody A33, specifically recognizing the epithelial cells of the colon. 394 proteins have been identified, belonging to various categories of proteins, some being common to those isolated from cultures of human cell lines of urine and from cultures of murine mast cell lines, thereby revealing a multifunctional role of the exosomes.

The importance of exosomes having been established, it is essential to provide effective and specific techniques for isolating the exosomes, allowing at the same time to be reliable, routinely usable and not requiring large volumes of biological samples.

### BRIEF SUMMARY

The invention concerns a method for isolating exosomes which comprises two successive separation steps based on the affinity separation technique. This sequential separation confers a high specificity to the method of the invention. Moreover, it enables it to be applicable to every sample of a biological liquid or fluid.

The method of the invention constitutes a tool which can be used routinely. It overcomes the obstacles to which those skilled in the art have been confronted to date, thereby opening a generalized access path to the characterization of exosomes.

The method of the invention comprises at least the following two successive steps:

a) a first step of affinity purification, applied on a biological liquid, using at least one specific anti-ligand of a generic ligand of the exosomes, so as to obtain a population P of exosomes, said exosomes being separated from said anti-ligand, and

b) a second step of affinity purification, applied on the population P of exosomes, using at least one specific anti-ligand of a ligand characteristic of a subpopulation SP of exosomes, so as to obtain said subpopulation SP of exosomes, said exosomes being separated or not from said anti-ligand.

Before exposing the invention in details, some terms employed in the present text for characterizing the invention are defined hereinafter.

The exosomes belong to a fraction of nano-vesicles secreted by the cells in the biological liquids. Structurally, these are vesicles having a lipid bilayer comprising proteins and sugars at their surface. They are defined by their size which varies from 30 to 200 nm, more particularly by a size of at least 40 nm, or even at least 50 nm, at of at most 150 nm, or even at most 120 nm and even at most 100 nm.

The terms biological liquid and biological fluid are employed interchangeably. A biological liquid is produced by a human or an animal, whether healthy or diseased, diagnosed or not. It is collected or punctured in the human or the animal, directly or indirectly. By indirectly, it is understood that it is possible to collect, in the human or the animal, cells or a cellular tissue which are cultured in a suitable medium in which said cells will excrete exosomes and all or part of which will be collected to be subjected to the isolation method of the invention. As non-limiting examples, we can mention cell supernatants, stool and bone marrow collections.

By affinity purification technique, it is understood a technique based on a specific interaction or recognition between an exosomal ligand carried by the exosome and an anti-ligand. This recognition may be of immunological nature and leads to an immune complex, such as the antigen/antibody, epitope/antibody, epitope/paratope, antigen/paratope interactions . . . , it is then question of immunopurification. This recognition can also be of any other nature, for example, of covalent nature. This technique allows isolating, from the sample, the exosome still bound to said anti-ligand or the exosome separated from said anti-ligand, for example after elution. A preferable implementation of this technique according to the invention comprises directly or indirectly attaching said anti-ligand on a solid support, for example magnetic beads, membranes, chromatography matrices, microplates, or still microfluidic apparatuses.

In the case of indirect attachment, the attachment of the anti-ligand to the solid support may be implemented by using a magnetic bead as an intermediate between the solid support, for example a membrane, and the anti-ligand. To do so, the activation of a magnet behind the solid support will allow attaching the magnetic bead to the solid support, and the deactivation of said magnet will allow separating, from the solid support, the exosomes attached to the anti-ligand, in turn attached to the magnetic bead.

By normal cell, it is understood a cell which has no detectable markers or a detectable level of markers which are characteristic of an abnormal condition of the cell. This definition includes stem cells, in particular mesenchymal, neural and hematopoietic cells, which secrete exosomes characteristic of a non-differentiated condition. In contrast, a cell is considered to be abnormal, when it has one or several detectable marker(s), or a detectable level of markers which are characteristic of a different condition from that of a normal cell, and in particular a pathological condition of the cell or a condition likely to evolve toward a pathological condition. This definition includes in particular cancerous, adenomatous, infectious, inflammatory, immunitarily stimulated, sunburn cells, and more generally, cells attacked by any type of stimulus.

As indicated before, despite their very small size, the exosomes have the advantage of accumulating very numerous proteins which constitute their traceability, and which are the quintessence of the cells that secrete them, in particular, the nature of the cellular tissue, the normal or abnormal condition of the cell . . . .

The method of the invention includes a first step of affinity purification which allows isolating a population P of exosomes, which is based on the existence of specific markers of the exosomes, likely to be present or exposed at the surface thereof, at a stage of their development. These markers, which will also be called generic ligands, are antigens, receptors, growth factors, as well as any other particle or molecule, and any fraction thereof, able to be specifically recognized by an anti-ligand.

Thus, the first step implements at least one anti-ligand which specifically recognizes at least one of the above-mentioned markers or ligands. Depending on the specificity of the ligand toward the exosomes, and in order to improve the effectiveness of this step, it is possible to use two anti-ligands or more, each of which being specific to two generic ligands or more, respectively, these generic ligands being specific to the exosomal population. According to a variant of the invention, the first step includes only one single purification, during which one or several of the aforementioned anti-ligands is/are used. According to another variant of the invention, the first step may comprise two consecutive sub-steps or more, each of which involving one or several specific anti-ligand(s) of one or several generic ligand(s), respectively.

As is illustrated in the examples, the first step leads to obtaining a population P of exosomes which are separated from said specific anti-ligand(s) of generic ligand(s) of the exosomes. Preferably, this separation is carried out by elution, under conditions which fall within the competence of those skilled in the art.

This first step a) may be applied on any biological liquid as defined above.

Beforehand, this liquid may have been treated. Thus, without departing from the scope of the invention, the biological liquid may be pretreated by a step of physical separation, by size, allowing to isolate a fraction of the biological liquid which does not contain molecules or particles of a size larger than 800 nm, preferably 500 nm, which will be then subjected to the first step a).

This step of physical separation may be carried out by any suitable technique such as those selected among the techniques of centrifugation and/or filtration, such as serial filtration, ultrafiltration, size exclusion chromatography, and the combinations of these techniques.

One advantage of this step of physical separation prior to the successive steps of affinity purification lies in that it allows enriching the sample before implementing said steps of affinity purification, thereby resulting in exosomes with an even higher purification.

Upon completion of the first step a), a population P of exosomes is obtained and then engaged in the second step b) of the method of the invention.

The second step of affinity purification of the method of the invention allowing to isolate a subpopulation SP of exosomes, from the population P of exosomes, is based on the existence of specific markers particular to, or characteristic of some exosomes, said markers being likely to be present or exposed at the surface thereof, at a stage of their development. These markers, which will be called the particular ligands, as opposed to the generic ligands, are antigens, receptors, growth factors, as well as any other particle or molecule, and any fraction thereof, capable of being specifically recognized by an anti-ligand.

The second step b) implements at least one anti-ligand specifically recognizing at least one of the aforementioned particular markers or ligands. Depending on the specificity of the ligand towards the exosomes, and in order to improve the effectiveness of this step, it is possible to use two anti-ligands or more, each of which being, respectively, specific to two particular ligands or more.

As is illustrated in the examples, and depending on the technology in which the exosomes isolated according to the invention are then involved, the second step leads to obtaining a subpopulation SP of exosomes which are bound to said specific anti-ligands of said ligand(s) characteristic of a subpopulation SP of the exosomes, or which are separated from said anti-ligands. In this last case, the obtained exosomes are released from every support and are soluble. Preferably, the separation of the exosomes from the anti-ligands is carried out by elution, under conditions which fall within the competence of those skilled in the art.

According to a variant of the invention, the second step b) includes only but one purification, during which one or several of the aforementioned anti-ligands are is/used. The purification of this second step b) may be carried out into several sub-steps involving one or several anti-ligand(s), contributing to the isolation of the same subpopulation SP of exosomes, which will therefore be more and more specific.

According to another variant of the invention, the method of the invention comprises at least one third step c) of affinity purification, this step being applied on said subpopulation SP and using at least one specific anti-ligand of a ligand characteristic of a sub-population SP′ of exosomes, the subpopulation SP′ being included in the subpopulation SP, so as to obtain said subpopulation SP′. Like the second step b), this third purification step c) may comprise sub-steps contributing to the isolation of the same subpopulation SP′ of exosomes.

Of course, one or several additional subsequent step(s) of affinity purification may further complete the method of the invention. In this case, the exosomes will be separated from said anti-ligands during the affinity purifications preceding the last step of affinity purification, the exosomes may then be separated or not from said anti-ligands during the last purification step.

The subpopulation SP and the subpopulation SP′ are particular populations of exosomes. Depending on the particular anti-ligands used, the subpopulation SP or the subpopulation SP′ may gather, as example, exosomes coming from the same organ, the same cellular tissue, or the same type of cells. The tissue or original cells may be normal or abnormal.

In practice and before illustrating the implementation and the advantages of a method of the invention, in the examples that follow, the population P, derived from the first step a), may be subjected to the second step b) of the method, in the presence of one or several specific anti-ligand(s) of ligands characteristic of a pathological organ, for example a prostate tumor, in order to isolate a subpopulation SP of exosomes characteristic of a prostate tumor. In another implementation, the population P may be subjected to the second step b), in the presence of one or several specific anti-ligand(s) of ligands characteristic of an organ, for example the prostate, without any indication of the normal or pathological condition of the organ. In order to refine the isolation method, this subpopulation SP may be subjected to a third step c) with specific anti-ligands of marker ligands of abnormal cells, so as to enable the isolation of a subpopulation SP′ of characteristic exosomes of a tumor of the prostate. It is also possible to consider that this subpopulation SP′ is obtained by treating a population P derived from the first step a), in a second step b) in the presence of specific anti-ligands of marker ligands of abnormal cells so as to isolate a subpopulation SP of exosomes characteristic of abnormal cells; then, this subpopulation SP is subjected to a third step c) with specific anti-ligands of ligands characteristic of the prostate.

The generic ligand(s) and/or the characteristic ligand(s) may be selected among polypeptides, proteins, antigens, receptors, enzymes, growth factors, glycolipids, polysaccharides, and the specific anti-ligands of said ligands are antibodies, fragments of antibodies such as the fragments F(ab′)2, scFv, antibody analogs, lectins, aptamers, peptides. According to the invention, the or at least one of the generic ligands is different from the or at least one of the characteristic ligands.

By <<antibody analogs>>, it is meant biological and/or chemical components which have the same binding capacities as the antibodies or fragments of antibodies or similar binding capacities. In particular, the analogs of antibodies include small proteins which, like the antibodies, are able to bind to a biological target thereby allowing to detect it, to capture it or simply to target it within an organism or within a biological sample. The fields of applications of these analogs of antibodies are almost as wide as the ones of the antibodies. As example, mention may be made to the Nanofitines™, which are small proteins commercialized by the AFFILOGIC company.

More particularly, the generic ligand is selected among the proteins of the tetraspanins family, such as the tetraspanins CD63, CD9, CD81, the proteins involved in the adhesion such as lactadherin (or MFGE-8), ICAM-1; proteins involved in the transport and the membrane fusion of the Rab-GTPases family, such as Rab5 and Rab7 and annexins and molecules of the major histocompatibility complex (MHC) such as MHI, MHII.

Preferably, the characteristic ligand of a subpopulation is selected among PSCA, annexin A3, PSMA, caveolin, B7H3, proteins of endogenous retroviral origin such as the envelope proteins.

Furthermore, the invention lies in a use of a method as previously described for characterizing and/or quantifying exosomes. As indicated before, the exosomes isolated according to the invention may be separated from the anti-ligand(s) involved in step b) of affinity purification or in the last step of affinity purification if other steps of affinity purifications are performed.

The exosomes isolated according to the method of the invention, separated from every support and soluble, are particularly useful in the following applications, given as illustrative and non-restrictive:
Nanotechnologies/Nanosystems
Sequencing of the exosomal content/genotyping
Therapy: nanocarrier and delivery of therapeutical targets
Studies in vitro: cellular messengers.

The method of the invention may also be applied for the diagnosis and prognosis of a pathology and/or of the clinical stage of a pathology, but also for monitoring the evolution of a pathology, whether treated or not, in a diseased human or animal, or for monitoring the effectiveness of the treatment of this pathology, in a diseased human or animal.

The pathology may be chronic or acute, of infectious or non-infectious origin. In one use of the method, the pathology is an adenoma, a cancer, an inflammation, a septicemia, a neurological disease such as Alzheimer's, Parkinson's diseases, the multiple sclerosis and the prion diseases, or a pregnancy pathology such as pre-eclampsia.

The treatment may be a medicinal treatment, a radiotherapy or a graft.

### BRIEF DESCRIPTION OF THE DRAWINGS

The invention is illustrated in the following examples where it is applied on different biological liquids of healthy and diseased patients, namely patients having a prostate cancer, the anti-ligands used at the second step being characteristic of the prostate. These examples refer to the following figures:

FIG. 1 represents the detection of exosomes in ng/μl by the ExoTEST sandwich assay Rab5/CD63 in the subpopulations immunopurified by a method of the invention, with the generic marker CD63 and the specific markers PSMA (Prostate-Specific Membrane Antigen) and caveolin, obtained from pools of serum of healthy and diseased subjects.

FIG. 2 represents the detection of exosomes in ng/μl by the ExoTEST sandwich assay Rab5/CD63 in subpopulations immunopurified by a method of the invention, with the generic marker CD63 and the markers AnxA3, PSCA (Prostate Stem Cell Antigen) and B7H3 (marker of tumoral epithelial cells and prostate cancer), from pools of plasma CaP of diseased subjects.

FIG. 3 is the result of a TEM observation of the exosomes on beads of the subpopulation CD63/PSMA isolated from pools of sera.

FIG. 4 is the result of a TEM observation of the exosomes on beads of the subpopulation CD63/AnxA3 isolated from pools of sera.

FIG. 5 is the result of a TEM observation of the exosomes of the subpopulation CD63/AnxA3 isolated from pools of sera, after elution (or desorption).

FIG. 6 represents the detection of exosomes in ng/μl by the ExoTEST sandwich assay Rab5/CD63 in subpopulations immunopurified by a method of the invention, with the generic marker CD63 and the markers PSMA, caveolin and AnxA3 for 2 sera P1 and P2 of subjects having a prostate cancer, tested individually.

FIG. 7 represents the detection of exosomes in ng/μl by the ExoTEST sandwich assay Rab5/CD63 in subpopulations immunopurified by a method of the invention, with the generic marker CD63 and the markers CD9, PSMA, and AnxA3 for the pools of plasma CaP and EFS.

FIG. 8 represents a comparison between the detection of the exosomes in a subpopulation CD63/AnxA3 of plasmatic origin (EFS and CaP) by the Nanosight technique and the one carried out by ExoTEST.

FIG. 9 represents a comparison between the detection of the exosomes in a subpopulation CD63/PSMA of plasmatic origin (EFS and CaP) by the Nanosight technique and the one carried out by ExoTEST.

FIG. 10 represents the detection of exosomes in ng/μl by the ExoTEST sandwich assay Rab5/CD63 in subpopulations immunopurified by a method of the invention, with the generic marker CD63 and the specific marker PSCA for plasmas of subjects having a prostate cancer (n=6) and healthy subjects (n=3).

### DETAILED DESCRIPTION AND EXAMPLES

### Example 1

### Application of the Method of the Invention on a Sample of Blood (Serum or Plasma) or Urine, in Order to Isolate Exosomes Characteristic of Tumoral Prostatic Cells

1) Equipment

1.1) Samples of Biological Liquids

Samples of Diseased Subjects

Pool of Sera CaP:

The purification method has been carried out by means of a pool of sera (V=2.5 ml) composed of 6 samples of patients having a prostate cancer at different stages of the disease with Gleason scores measuring the aggressiveness of the cancerous cells, ranging from 6 to 9 (on a scale of 2 to 10).

Individual Sera CaP:

A study on 2 individual sera derived from the pool of samples hereinabove has been carried out from a volume of serum reduced to V=1.2 ml, namely a test sample 2 times smaller. It comprises 2 sera coming from patients (P1 and P2) having a Gleason score of 7 and 8, respectively.

Pool of Plasmas CaP:

The purification method has been applied on a pool of plasmas (V=2.5 ml) constituted by 4 samples of patients having a prostate cancer in the metastatic phase.

Pool of Urines CaP:

The purification method has been applied on a pool of urines of patients having a prostate cancer with a Gleason score of 7, after a post-digital rectal examination (post-DRE) massage (CaP), and patients having a benign prostatic hyperplasia (BPH).

Samples of Healthy Subjects

Pool of Sera EFS:

Serous samples coming from 6 healthy donors of the French Blood Establishment (EFS) have allowed constituting a pool of serum EFS (V=2.5 ml) as a control group for the study.

Pool of Plasmas EFS:

Plasmatic samples coming from 6 healthy donors of the EFS have allowed constituting a pool of plasmas EFS (V=2.5 ml) as a control group for the study.

1.2) Used Antibodies

They are listed in Table 1 below.

TABLE 1

| Antibody | Clone | Type | Target | Marker |
|---|---|---|---|---|
| anti-CD63¹ | MX-49, | Mouse monoclonal | CD63 | Generic |
| | 129, 5 | | | |
| anti-CD9² | MEM-61 | Mouse monoclonal | CD9 | Generic |
| anti-PSM¹ | K1H7 | Mouse monoclonal | PSMA | Prostate |
| | | | | cancer |
| anti-PSCA³ | 5C2 | Mouse monoclonal | PSCA | Prostate |
| | | | | cancer |
| anti-AnxA3⁴ | 13A12G4 | Mouse monoclonal | Annexin A3 | Prostate |
| | | | | cancer |
| anti-B7H3¹ | 4396 | Mouse monoclonal | B7H3 | Aggressive |
| | | | | cancer |
| anti- | N-20 | Rabbit polyclonal | Caveolin | Cancer |
| Caveolin¹ | | | | |

¹supplied by Santa Cruz Technology
²supplied by Novus Biological
³supplied by Sigma Aldrich
⁴produced by the bioMérieux company as described in FR2968767A1

1: supplied by Santa Cruz Technology
2: supplied by Novus Biological
3: supplied by Sigma Aldrich
4: produced by the bioMérieux company as described in FR2968767A1

2) Preparation of the Anti-Ligands

The aforementioned antibodies are coupled to magnetic beads covered with streptavidin (Dynabeads® M-280 Streptavidin).

A step of biotinylation of the antibodies is carried out beforehand with the commercial kit, One-step Antibody Biotinylation, commercialized by Miltenyi Biotec, according to the recommendations of the supplier.

A volume of 150 μl of beads MP-280 (namely 10⁸ beads) is collected then washed for 5 minutes with 500 μl of a buffer+0.5% Tween. The tube is placed on a magnetized support in order to eliminate the supernatant. 500 μl of the human monoclonal antibodies anti-CD63, anti PSMA, anti-AnxA3, anti-caveolin or anti-CD9 diluted at the concentration of 20 μg/ml in the buffer+0.5% Tween are added and incubated for 30 minutes under rotary stirring. In order to block the free sites, a solution of biotin at 10 mM is added and incubated for 30 minutes under rotary stirring. Afterwards, the beads are washed 5 times for 5 minutes with 500 μl of buffer+0.5% Tween. The conjugate streptavidin beads MP-280/biotinylated antibody is ready to be brought into contact with the blood sample.

3) Pretreatment of the Sample

The serous or plasmatic sample is subjected to a prior ultracentrifugation treatment comprising two differential centrifugations and a filtration as follows.

The sample (volume of 2.5 ml) is centrifuged at 500 g for 10 minutes at +4° C. in order to eliminate the blood cells and the cellular debris, then at 16500 g for 20 minutes at +4° C. so as to subtract microparticles and apoptotic bodies from the fluid. A step of filtering on 0.45 μm is carried out in order to eliminate the extracellular vesicles and the protein aggregates of a size larger than 450 nm.

In turn, the urine sample is also subjected to a prior treatment of differential centrifugations, then of filtering on 0.45 μm. Afterwards, the urine is concentrated 5× on Vivaspin 20 (cut off 10 kD, Vivasciences).

4) Application of the First Immunopurification Step a) of the Method of the Invention in Order to Obtain a Population P

For this step, a tetraspanin generic ligand is targeted.

The used anti-ligand is an anti-tetraspanin antibody, more specifically an anti-CD63.

The first immunopurification by the anti-tetraspanin CD63 is carried out through 2 incubations of the pretreated blood sample. First, a batch is carried out by incubating a volume of 1.25 ml of the sample with the conjugate streptavidin beads/biotinylated antibody anti-CD63 for 3 hours at ambient temperature under rotary stirring. The remaining volume of 1.25 ml of pretreated serum is incubated in batch with the bioconjugate for one night under rotary stirring at ambient temperature. 5 washes for 5 minutes are carried out with 500 μl of buffer+0.5% Tween. The final elution step is carried out by adding 100 μl of an elution buffer (0.2M glycine, HCl, pH 2.2+1 mg/ml of BSA) after 2 minutes of incubation with the bioconjugate while gently vortexing for a few seconds. 14 μl of a neutralization buffer (Tris 2M, pH 9.5) are added to a first elution volume E1 of 100 μl. A second identical elution+neutralization E2 is carried out. The eluates E1 and E2 are mixed and a final elution volume of 228 μl is preserved at −80° C. till analysis.

Under the same conditions, this immunopurification step has been carried out with the anti-tetraspanin antibody, anti-CD81, on the pools of sera of diseased patients (CaP).

The pools of urines CaP and BPH are subjected to this first immunopurification step with the anti-tetraspanin CD63.

5) Application of the Second Immunopurification Step b) of the Method of the Invention in Order to Obtain a Subpopulation SP

For this step, the particular ligands are the PSMA, the caveolin and the annexin A3.

The used anti-ligands are the anti-PSMA, the anti-caveolin, the anti-annexin A3 antibodies.

This second step of specific immunopurification is carried out from the elution fractions P CD63 and P CD81, respectively, obtained at 4). A volume of 205 μl of the elution P is adjusted to a final volume of 1200 μl with the buffer and fractionated in three times 400 μl before being brought into contact with the bioconjugates beads/biotinylated antibody anti-PSMA, beads/biotinylated antibody anti-caveolin and beads/biotinylated antibody anti-annexin A3, for 3 h at ambient temperature under rotary stirring. 5 washes for 5 minutes are carried out with 500 μl of buffer+0.5% Tween. In the same manner as for the population P, the elution is carried out in two times with an obtained final elution volume of 228 μl preserved at −80° C. till analysis.

The following subpopulations SP have been obtained: CD63/PSMA, CD63/caveolin, CD63/AnxA3, CD81/PSMA, CD81/PSCA, CD81/AnxA3.

The pools of urines CaP and BPH are subjected to the second immunopurification step with the anti-PSMA antibody.

### Example 2

### Techniques for the Detection of the Exosomes Purified by the Method of the Invention

1) Immunodetection of the Exosomes by ELISA, ExoTEST® (Supplied by HansaBiomed)

In order to detect the presence of exosomes in the fractions purified by the previously described method of the invention, the ExoTEST test commercialized by the HansaBiomed company has been used. It comprises an ELISA sandwich microplate assay test which uses a monoclonal antibody directed against the protein Rab5 (belonging to the RabGTPases family) for capture and a monoclonal antibody anti-CD63 for detection. The sandwich format allows for the specific capture of the exosomes by reducing the detection of contaminant proteins. Furthermore, the test allows quantifying the exosomes from biological samples and from preparations purified and enriched with exosomes, thanks to the presence of a calibration standard included in the kit. The assay of the samples has been carried out according to the recommendations of the supplier.

2) Detection of the Exosomes by the Physical Method, NTA (Nanoparticle Tracking Analysis)

The NanoSight company commercializes an analysis instrument, the LM10-HS, which allows measuring and characterizing all types of nanoparticles of a size comprised between 10 nm and 1 μm, within a polydisperse sample. Using a 405 nm laser, the nanoparticles are excited and their Brownian motion is monitored by means of an optical microscope and filmed with a camera. The software provided with the apparatus (Nanosight 2.0) allows obtaining an analysis of the size and concentration of the different particles present in the sample.

A detection threshold has been determined from the measurements carried out from 6 injections of the PBS1× buffer filtered 2 times beforehand on 0.22 μm. This threshold corresponds to the average of the values+3 times the standard deviation, namely 0.42×10⁸ particles/mL.

At the beginning of each manipulation, the quality of the PBS1× buffer (filtered extemporaneously on 0.1 μm) used for the dilution of the samples is controlled. The blank value should not exceed the detection threshold.

The fractions immunopurified by the method according to the invention have been diluted to 1/50^{th} in PBS1× for the analysis. The fractions derived from the ultracentrifugation pretreatment [cf. Example 1, 3)] are diluted to 1/500^{th} in PBS1×.

For each sample, the coefficient of variation (CV) is calculated on the measurements of concentration and size (mode and mean) obtained after 5 to 6 injections. These measurements allow assessing the reproducibility of the NTA analysis of the samples.

3) Detection of the Exosomes by Transmission Electron Microscopy (TEM)

The direct observation of the exosomes in suspension after negative staining is carried out by transmission electron microscopy. There is observed a) the exosomes coupled to the beads and b) the exosomes alone, after elution.

### Example 3

### Application in the Detection of Immunopurified Exosomes Derived from a Pool of Sera According to the Invention

The different detection techniques exposed in Example 2 are applied in the present example on the subpopulations of exosomes CD63/AnxA3 and CD63/PSMA, derived from the pool of sera of Example 1 after the treatments 4) and 5) of Example 1.

1) Detection with the ExoTEST®

Starting from a pool of sera of patients having a prostate cancer CaP and a pool from healthy donors (EFS), the detection of the circulating exosomes isolated by generic and specific sequential immunoaffinity is carried out by means of the ExoTEST sandwich ELSIA assay Rab5/CD63. A test sample corresponding to ¼ the volume of each obtained exosomal isolation fraction is assayed by ExoTEST®.

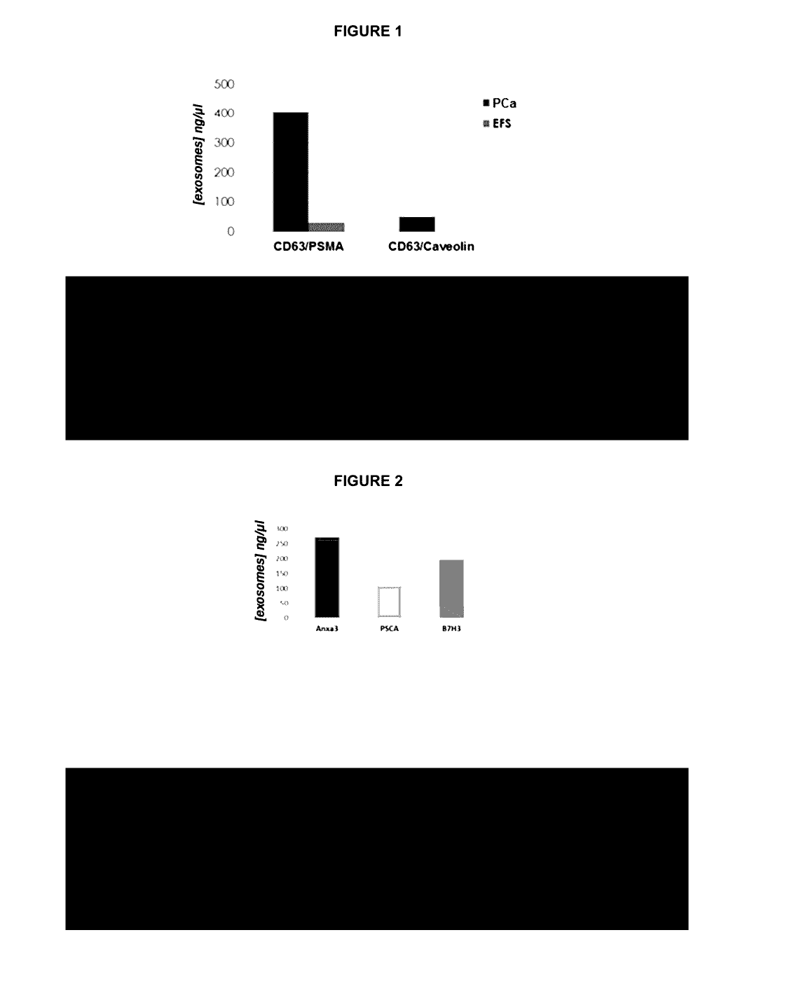

FIGS. 1 and 2 indicate the concentration of exosomes in ng/μl obtained for the different markers tested during the second immunopurification.

FIG. 1 highlights the detection of serous exosomes in the following fractions:
The population CD63/PSMA corresponds to a subpopulation of exosomes derived from a double immunopurification method according to the invention, by applying a step a) of the method on the pool of sera CaP (subjects having a prostate cancer) and on the pool of sera EFS (healthy subjects), using an anti-ligand directed against the generic ligand CD63, then by applying a step b) on the thus isolated population, using an anti-ligand directed against the specific marker of the prostate cancer PSMA
The population CD63/Caveolin corresponds to a subpopulation of exosomes derived from a double immunopurification method according to the invention, by applying a step a) of the method on the pool of sera CaP (subjects having a prostate cancer), using an anti-ligand directed against the generic ligand CD63, then by applying a step b) on the thus isolated population, using an anti-ligand directed against the specific marker of the caveolin prostate cancer.

Higher concentrations of exosomes are detected for the pool of sera CaP of prostate cancer, in comparison with the pool of sera coming from healthy donors.

FIG. 2 highlights the detection of serous exosomes in the following fractions:

The subpopulations CD63/AnxA3, CD63/PSCA and CD63/B7H3 of exosomes are obtained upon completion of a method of double immunopurification according to the invention, by applying a step a) of the method on the pool of sera CaP (subjects having a prostate cancer), using an anti-ligand directed against the generic ligand CD63, then by applying a step b) on the thus isolated population, using an anti-ligand directed against the specific markers AnxA3, PSCA and B7H3, respectively.

The specificity of the isolation method of the invention has been verified through a Luminex sandwich immunoassay AnxA3 of the subpopulation CD63/AnxA3. This assay is very sensitive, it has a calculated limit of detection of 1.1 pg/ml. By this assay, an AnxA3 concentration of 50 pg/mL has been measured in the subpopulation CD63/AnxA3.

The following Table 2 gives the concentration values of the exosomes detected by ExoTEST® applied on the subpopulations CD81/PSMA, CD81/PSCA, CD81/AnxA3:

TABLE 2

| | Subpopulation | | |
|---|---|---|---|
| | CD81/PSMA | CD81/PSCA | CD81/AnxA3 |
| Concentration of | 763.44 | 615.33 | 690.02 |
| exosomes (ng/μl) | | | |

2) Detection by NTA

The analysis of the subpopulations SP CD63/AnxA3 and SP CD63/PSMA derived from the pool of sera CaP, performed by NTA indicates the concentration of exosomes as well as the size of the major and mean peak in the purified samples figuring in Table 3 below.

TABLE 3

| | | | Average | | Size of the | |
|---|---|---|---|---|---|---|
| | Particles | CV | size of the | CV | maximum | CV |
| SP | ×10⁹/ml | % | peaks (nm) | % | peak (nm) | % |
| CD63/AnxA3 | 16.4 | 24 | 125 | 1.7 | 92 | 4.5 |
| CD63/PSMA | 24.6 | 30 | 77 | 3 | 99 | 4 |

There is observed a good reproducibility of the size distribution profiles with major peak sizes, for each of the studied markers, comprised between 90 and 100 nm and coherent with the size described for the exosomes.

3) Detection by TEM

A morphological characterization of the vesicles isolated by transmission electron microscopy has been performed.

a) Observation of the Exosomes Attached on the Beads:

The captured vesicles are observed directly on the beads (Dynabeads® M-280 streptavidin) conjugated to the biotinylated antibodies anti-AnxA3 and anti-PSMA. Thus, after a first immunopurification of the serous exosomes by means of the anti-CD63, the eluate is incubated with coated beads either with the anti-AnxA3, or with the anti-PSMA, washed and taken up in PBS.

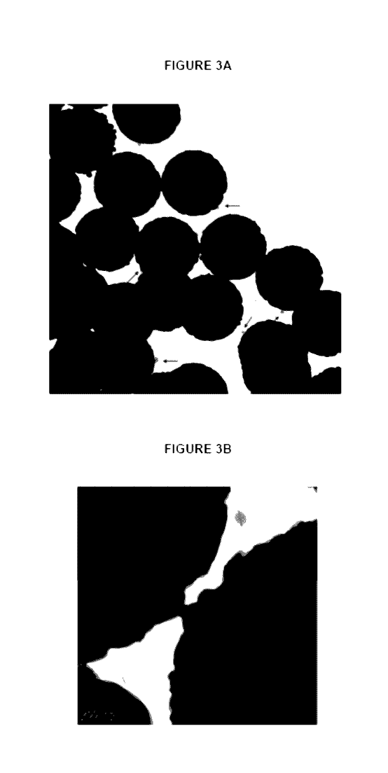

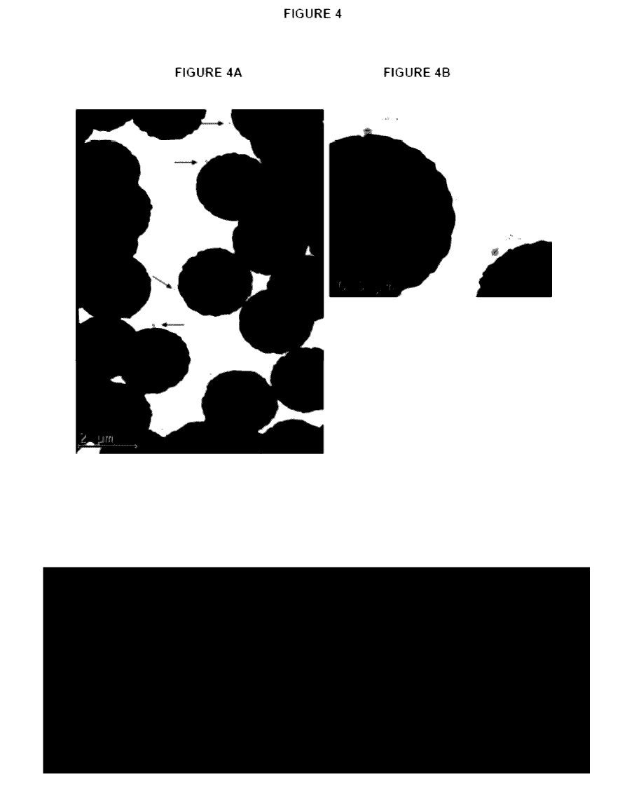

The result of this observation is illustrated in the images below of FIG. 3 (FIGS. 3A and 3B) for the SP CD63/PSMA and of FIG. 4 (FIGS. 4A and 4B) for the SP CD63/AnxA3, in which the capture of small vesicles at the surface of the magnetic beads is illustrated.

b) Observation of the Exosomes Alone after Elution:

A volume of 7.5 mL of the pools of sera has been subjected to a double immunopurification according to the invention by using the antibodies anti-CD63 for the first step and anti-AnxA3 and anti-PSMA, respectively, for the second step. Each of the subpopulations SP, CD63/AnxA3 and CD63/PSMA has been subjected to an elution by 2×60 μl of a glycine buffer 0.2M, under a pH of 2.5, then neutralized.

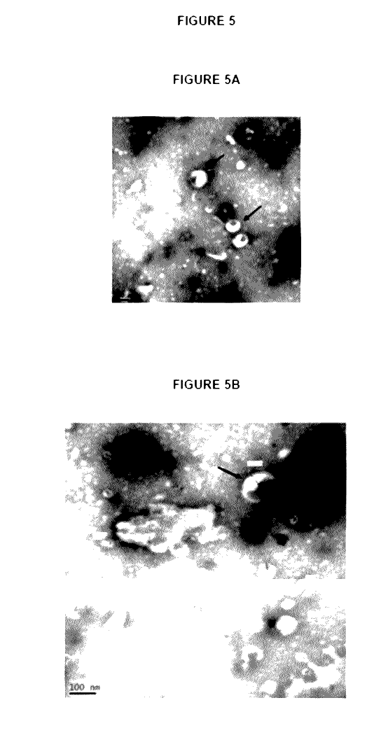

The result of this observation is illustrated in FIG. 5 (FIGS. 5A and 5B). The <<cup-shaped>> morphology and the size varying from 50 to 120 nm of the observed vesicles are typical of exosomes, thereby demonstrating the effectiveness of the method of the invention.

### Example 4

### Application in the Detection of the Immunopurified Exosomes Derived from Individual Sera According to the Invention

The detection technique with the ExoTEST® exposed in Example 2 is applied in the present example on the subpopulations of exosomes CD63/AnxA3 and CD63/PSMA, derived from the individual sera CaP (diseased subjects) 1.1 of Example 1 after the treatments 4) and 5) of Example 1.

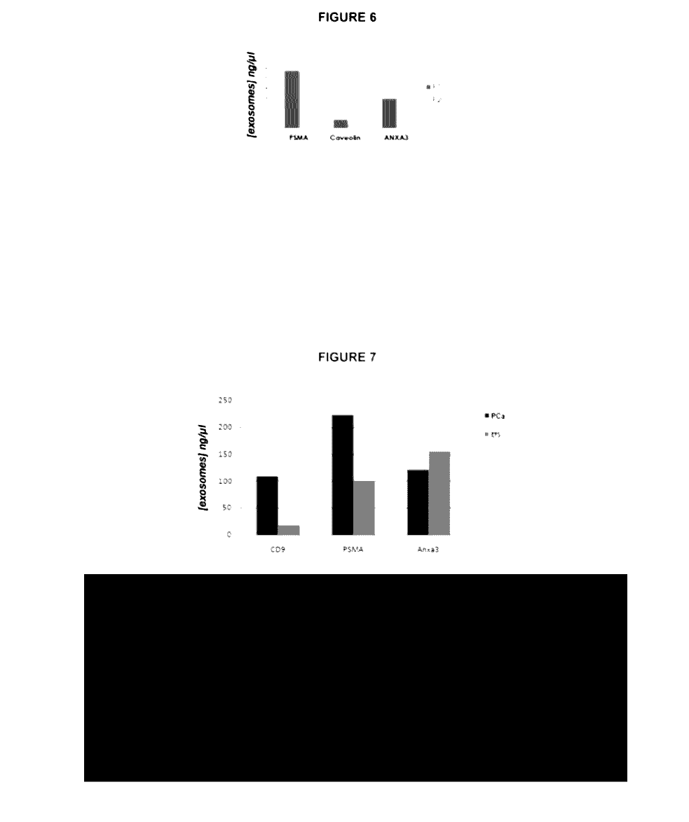

FIG. 6 indicates the concentration of exosomes obtained for the individual sera P1 and P2 derived from the pool of sera of patients having a prostate cancer. The populations PSMA, Caveolin and AnxA3 of exosomes are obtained subsequently to a double immunopurification method according to the invention, by applying a step a) of the method on the sera P1 and P2, using an anti-ligand directed against the generic ligand CD63, then by applying a step b) on the thus isolated population, using an anti-ligand directed against the specific markers PSMA, Caveolin and AnxA3, respectively.

### Example 5

### Application in the Detection of Immunopurified Exosomes Derived from a Pool of Plasmas According to the Invention

The detection techniques with ExoTEST® and by NTA exposed in Example 2 are applied in the present example on the subpopulations of exosomes CD63/AnxA3 and CD63/PSMA, derived from the pool of plasmas CaP (diseased subjects) and from the pool of plasmas EFS (healthy subjects) of Example 1 after the treatments 4) and 5) of Example 1.

1) Detection with the ExoTEST®

Starting from a pool of plasmas of patients having a prostate cancer and from a pool of healthy donors (EFS), the detection of the circulating exosomes isolated by the method of the invention is carried out by means of the ExoTEST sandwich ELISA assay Rab5/CD63. The different obtained exosomal isolation fractions are diluted to ½ in PBS1× and assayed by ExoTEST.

FIG. 7 indicates the concentration of exosomes in ng/μl obtained in the following fractions:

The population CD63/PSMA corresponds to a subpopulation of exosomes derived from a double immunopurification method according to the invention, by applying a step a) of the method on the pool of plasmas CaP (subjects having a prostate cancer) and the pool of plasmas EFS (healthy subjects), using an anti-ligand directed against the generic ligand CD63, then by applying a step b) on the thus isolated population, using an anti-ligand directed against the specific marker of the prostate cancer PSMA
The population CD63/AnxA3 corresponds to a subpopulation of exosomes derived from a double immunopurification method according to the invention, by applying a step a) of the method on the pool of plasmas CaP (subjects having a prostate cancer) and the pool of plasmas EFS (healthy subjects), using an anti-ligand directed against the generic ligand CD63, then by applying a step b) on the thus isolated population, using an anti-ligand directed against the specific marker of the prostate cancer Annexin A3.

Higher concentrations of exosomes are observed for the pool of plasmas CaP, in comparison with the pool of plasmas of the healthy donors for the PSMA marker.

Interestingly, there is observed a concentration difference of exosomes between the pool of plasmas of healthy donors and the pool of plasmas CaP for the marker AnxA3, the first concentration being higher than the second one.

2) Detection by NTA

The analysis of the SP CD63/AnxA3 and CD63/PSMA derived from the pool of plasmas CaP and from the pool of plasmas EFS, performed by NTA, indicates the concentration of exosomes as well as the size of the major and mean peak in the purified samples figuring in Table 4 below.

TABLE 4

| | | | Average | | Size of the | |
|---|---|---|---|---|---|---|
| | Particles | CV | size of the | CV | maximum | CV |
| SP | ×10⁹/ml | % | peaks (nm) | % | peak (nm) | % |
| CD63/AnxA3 | 3.1 | 10.8 | 140 | 32.4 | 108 | 14.1 |
| EFS | | | | | | |
| CD63/AnxA3 | 2.7 | 12.8 | 124 | 2.8 | 85 | 7.3 |
| CaP | | | | | | |
| CD63/PSMA | 8.5 | 34.8 | 163 | 19.9 | 122 | 15.5 |
| EFS | | | | | | |
| CD63/PSMA | 14.2 | 15.9 | 176 | 7.5 | 133 | 14.4 |
| CaP | | | | | | |

The size of the major peaks is coherent with the described size of the exosomes.

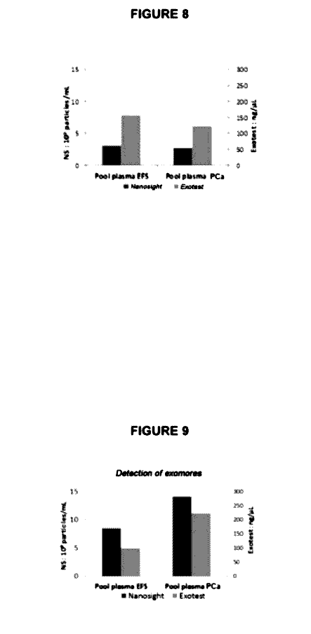

FIGS. 8 and 9 illustrate a comparison between the two techniques ExoTEST and NTA, respectively for the subpopulations SP CD63/AnxA3 and SP CD63/PSMA. There is observed an adequacy between these two detection techniques.

### Example 6

### Application in the Detection of the Immunopurified Exosomes Derived from Plasmas According to the Invention

The detection technique with the ExoTEST® exposed in Example 2 is applied in the present example on the subpopulations of exosomes CD63/PSCA, derived from plasmas of patients having a prostate cancer and from healthy subjects, respectively.

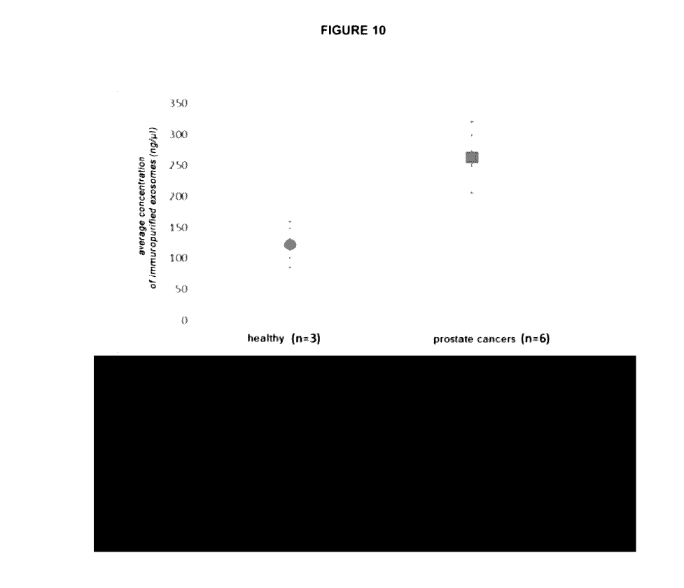

FIG. 10 indicates the average concentration of exosomes in the subpopulations SP CD63/PSCA isolated for the groups of healthy patients (n=3) and for the groups of patients having a prostate cancer (n=6), respectively. There is observed a higher level of plasmatic exosomes in the diseased patients than in the healthy patients.

### Example 7

### Application in the Detection of the Immunopurified Exosomes Derived from a Pool of Urines According to the Invention

The immunopurified fractions below are assayed by the ExoTEST ELISA assay. The results are presented in Table 5 below.

TABLE 5

| | CD63/PSMA |
|---|---|
| | ELISA ExoTEST |
| IP2 | [exosomes] ng/ml |
| Urine Pool BPH | 513 |
| Urine Pool CaP | 1366 |

A two times higher concentration of exosomes is detected for the CaP pool versus the BPH pool. Hence, urinary exosomes presenting the surface prostatic marker PSMA are twice as many in the urine of patients having a CaP.

### Example 8

### Specific Assay of the Total Prostate Specific Antigen (tPSA) Marker

The tPSA has been assayed in the pools of sera CaP and EFS as well as in the purified exosomal fractions in order to verify the quality of the exosomal fractions obtained after a double immunopurification.

For this purpose, an adaptation of the TPSA VIDAS sandwich assay, using the monoclonal antibody 12C11C3 for capture and the biotinylated antibody 11E5C6 for detection, has been made in the Luminex format. Using microspheres in suspension, the technique allows detecting and quantifying several biomolecules in the same sample of a small volume with a high sensitivity.

Magnetic beads of a 5.6 μm diameter, presenting a spectral address based on their red/infrared content have served as a support for the assay. An amount of 9 μg of the capture antibody 12C11C3 has been grafted at the surface of the magnetic beads (Bio-Rad, Bio-Plex Pro Magnetic COOH Beads Amine Coupling Kit) in according to the instructions of the supplier.

For the assay of the serous and plasmatic pools, the samples are diluted to the ⅕^{th} in a TBST buffer. For the purified exosomal fractions, the tPSA assay is carried out with ⅛ the volume of the eluted exosomal fraction.

The samples are incubated in a 96-well plate (Bio-Rad, 171025001) in the presence of 5000 beads coupled to the capture antibody for 2 hours at 37° C., 650 rpm, and protected from light. Between each step, the wells are washed 3 times in a TBST at 0.05%. The detection is carried out with 100 μl of biotinylated secondary antibody 11E5C6 at the concentration of 0.005 μg/ml for 1 hour at 37° C. under stirring. The revelation of the immune complex occurs by incubation of 100 μl of a streptavidin solution coupled to phycoerythrin (RPE) at the concentration of 2 μg/ml (Dako) for 30 minutes at 37° C. under stirring. The final step comprises re-suspending the immune complexes in 100 μl of TBS for a flow fluorimetry analysis performed by the Bio-Plex 200 (Bio-Rad) automaton. Each bead will undergo a dual excitation by a red laser (633 nm) for its identification and by a green laser (532 nm) for the quantification of the analyte by measuring the fluorescent conjugate.

The analytic detection limit of the tPSA assay developed on the Luminex reaches an excellent sensitivity of 1.1 pg/ml of tPSA.

The results of the assay in the serum CaP are presented in Table 6 below.

TABLE 6

| | [tPSA] in ng/ml | | |
|---|---|---|---|
| | Pool of | Purified fraction IP2 | |
| | sera | PSMA | AnxA3 |
| CaP | 2.98 | 0 | 0 |

The results of the Luminex tPSA assay show that the serous tPSA marker is detected at the concentration of 2.98 ng/ml in the pool of serum CaP. After purification of the serous pool, the tPSA marker is not, or very little, detected in the purified exosomal fractions. This result indicates the quality of the purified exosomal fraction obtained by the method of the invention, with the elimination of the soluble proteinic tPSA marker from the purified preparations.

### Example 9

### Repeatability and Reproducibility of the Method

The repeatability, that is to say the intra-series variability, and the reproducibility, that is to say the inter-series and inter-days variability, of the isolation method of the invention have been studied over 4 days.

The test is carried out for isolating the subpopulations CD63/AnxA3 and CD63/PSMA of exosomes from a pool of serum of diseased patients. The immunodetection of the exosomes is performed by ExoTEST and the results figure in Table 7 below.

TABLE 7

| Ligand | Statistics | Repeatability | Reproducibility |
|---|---|---|---|
| AnxA3 | Standard | 17.95 | 27.78 |
| | deviation | | |
| | CV (%) | 4.87 | 7.54 |
| PSMA | Standard | 129.89 | 94.80 |
| | deviation | | |
| | CV (%) | 24.63 | 18.62 |

There is observed a very good repeatability for the ligand AnxA3, with a CV of 4.87%. For this marker, the inter-days reproducibility also indicates a very good CV of 7.5%.

Slightly more significant but still perfectly acceptable variations are observed for the specific marker PSMA.

1. A method for isolating exosomes from a biological liquid, comprising at least the following two successive steps of affinity purification:
a) a first step using at least one specific anti-ligand of a generic ligand of the exosomes, so as to obtain a population P of exosomes, said exosomes being separated from said anti-ligand, and
b) a second step, applied on the population P of exosomes, using at least one specific anti-ligand of a characteristic ligand of a subpopulation SP of exosomes, so as to obtain said subpopulation SP of exosomes, said exosomes being separated or not from said anti-ligand.

2. The method according to claim 1, wherein the second step uses at least two anti-ligands, each of which being specific to a ligand respectively characteristic of a subpopulation SP1 and SP2.

3. The method according to claim 1, wherein the first step uses at least two anti-ligands, each of which being specific to a ligand respectively generic of the exosomes.

4. The method according to claim 1, wherein it comprises at least one third step c) of affinity purification, this step being applied on said subpopulation SP and using at least one specific anti-ligand of a ligand characteristic of a sub-population SP′ of exosomes, the subpopulation SP′ being included in the subpopulation SP, so as to obtain said subpopulation SP′.

5. The method according to claim 4, wherein the third step uses at least two anti-ligands, each of which being specific to a ligand respectively characteristic of a subpopulation SP′1 and SP′2.

6. The method according to claim 1, wherein the generic ligand of the exosomes and/or the ligand(s) characteristic of a subpopulation SP or SP′ of exosomes are selected among the ligands present at the surface of the exosomes.

7. The method according to claim 1, wherein the subpopulation(s) of exosomes is/are subpopulations of exosomes coming from the same organ.

8. The method according to claim 1, wherein the subpopulation(s) of exosomes is/are subpopulations of exosomes coming from the same tissue.

9. The method according to claim 1, wherein the subpopulation(s) of exosomes is/are subpopulations of exosomes coming from the same type of cells.

10. The method according to claim 1, wherein the subpopulation(s) of exosomes is/are subpopulations of exosomes coming from healthy tissue or cells or from abnormal tissue or cells.

11. The method according to claim 10, wherein the abnormal tissue or cells are tumoral.

12. The method according to claim 1, wherein the ligand is characteristic of a subpopulation SP of exosomes coming from the prostate.

13. The method according to claim 1, wherein, prior to the first step a), the biological liquid is treated through a step of physical separation, by size, which allows isolating a fraction of the biological liquid which does not contain molecules or particles of a size larger than 800 nm which will be subjected to the first step a).

14. The method according to claim 1, wherein, upon completion of step a), the exosomes are separated from the anti-ligand by elution.

15. The method according to claim 1, wherein, upon completion of step b), the exosomes are separated from the anti-ligand by elution.

16. A use of a method according to claim 1, for characterizing and/or quantifying exosomes.

17. The use according to claim 16, for the diagnosis and prognosis of a pathology and/or of the clinical stage of a pathology, and for monitoring the evolution of a pathology, whether treated or not, or for monitoring the effectiveness of the treatment of a pathology, in a human or an animal.

18. The use according to claim 17, wherein the pathology is chronic or acute, of infectious or non-infectious origin.

19. The use according to claim 17, wherein the treatment is a medicinal treatment, a radiotherapy or a graft.
